(19) United States
(12) Patent Application Publication
(10) Pub. No.: US 2010/0068263 A1
(43) Pub. Date: Mar. 18, 2010

(54) Method and Compositions for Stimulation of an Immune Response to TRP2 using a Xenogeneic TRP2 Antigen

(75) Inventors: Alan N. Houghton, New York, NY (US); Clarissa Naftzger, Belmont, CA (US); Setaluri Vijayasaradhi, Madison, WI (US)

Correspondence Address: Larson & Anderson, LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 12/622,103

(22) Filed: Nov. 19, 2009

Related U.S. Application Data
(63) Continuation-in-part of application No. 10/285,874, filed on Oct. 31, 2002.
Application No. 10/285,874 is a continuation-in-part of application No. 09/627,694, filed on Jul. 28, 2000, now abandoned.
Application No. 09/627,694 is a continuation-in-part of application No. 09/308,697, filed on May 21, 1999, now Pat. No. 6,328,969.
(60) Provisional application No. 60/036,419, filed on Feb. 18, 1997.

(51) Int. Cl. A61K 9/127; A61K 39/00; A61K 31/7052; A61P 37/04

(52) U.S. Cl. 424/450; 424/184.1; 514/44 R

(57) ABSTRACT

Tolerance of the immune system for endogenous TRP2 can be overcome and an immune response stimulated by administration of xenogeneic or xenoexpressed TRP2 antigen. For example, mouse TRP2, or antigenically-effective portions thereof, can be used to stimulate an immune response to the corresponding differentiation antigen in a human subject. Administration of xenogeneic antigens in accordance with the invention results in an effective immunity against TRP2 expressed by the cancer in the treated individual, thus providing a therapeutic approach to the treatment of cancers expressing TRP2, such as melanoma.

This application is a continuation-in-part of U.S. patent application Ser. No. 10/285,874, which is continuation-in-part of U.S. patent application Ser. No. 09/627,694, filed Jul. 28, 2000, which is continuation-in-part of U.S. patent application Ser. No. 09/308,697, filed May 21, 1999, which is a §371 National Phase of International Application No. PCT/US97/22669 filed Dec. 10, 1997. The application also claims benefit under 35 USC §119(e) of U.S. Provisional Application No. 60/036,419 filed Feb. 18, 1997. All of the aforementioned applications are incorporated herein reference.

### FIELD OF THE INVENTION

This application relates to a method and compositions for stimulation of an immune response to TRP2.

### BACKGROUND OF THE INVENTION

Most tumor immunity is mediated by recognition of self-antigens, antigens present in cancer cells that are also found in normal host tissue. Houghton, A. N., J. Exp. Med. 180: 1-4 (1994). This type of immunity is more akin to autoimmunity than to immunity in infectious diseases, where the response is directed at a truly foreign antigen, present in the pathogen but not in host tissue. Evidence of this can be found in the autoimmune sequalae that often follow the development of successful tumor immunity. Bowne, W. B., et al., J. Exp. Med. 190(11):1717-1722 (1999).

Differentiation antigens form one prototype of self-antigens in cancer immunity. Houghton, A. N., et al., J. Exp. Med. 156(6):1755-1766 (1982). Differentiation antigens are tissue-specific antigens that are shared by autologous and some allogeneic tumors of similar derivation, and on normal tissue counterparts at the same stage of differentiation. Differentiation antigens have been shown to be expressed by a variety of tumor types, including melanoma, leukemia, lymphomas, colorectal, carcinoma, breast carcinoma, prostate carcinoma, ovarian carcinoma, pancreas carcinomas, and lung cancers. Typically the expression of these antigens changes as a cell matures and can characterize tumors as more or less differentiated. For example, differentiation antigens expressed by melanoma cells include Melan-A/MART-1, Pmel17, tyrosinase, gp75 and gp100. Differentiation antigens expressed by lymphomas and leukemia include CD19 and CD20/CD20 B lymphocyte differentiation markers. An example of a differentiation antigen expressed by colorectal carcinoma, breast carcinoma, pancreas carcinoma, prostate carcinoma, ovarian carcinoma, and lung carcinoma is the mucin polypeptide muc-1. A differentiation antigen expressed by breast carcinoma is her2/neu. The her2/neu differentiation antigen is also expressed by ovarian carcinoma. Differentiation antigens expressed by prostate carcinoma include prostate specific antigen, prostatic acid phosphatase, and prostate specific membrane antigen (PSMA).

Unfortunately, in most cases, the immune system of the individual is tolerant of these antigens, and fails to mount an effective immune response. For the treatment of cancers where the tumor expresses differentiation antigens therefore, it would be desirable to have a method for stimulating an immune response against the differentiation antigen in vivo. It is an object of the present invention to provide such a method.

### SUMMARY OF THE INVENTION

It has now been found that the tolerance of the immune system for endogenous TRP2 can be overcome and an immune response stimulated by administration of xenogeneic TRP2 and TRP2 (including syngeneic TRP2) expressed in cells of different species. For example, mouse TRP2, or antigenically effective portions thereof, can be used to stimulate an immune response to the corresponding differentiation antigen in a human subject. Administration of xenogeneic or xenoexpressed antigens in accordance with the invention results in an effective immunity against TRP2 expressed by the cancer in the treated individual, thus providing a therapeutic approach to the treatment of melanomas expressing TRP2.

### BRIEF DESCRIPTION OF THE DRAWINGS

FIGS. 1A-C show Kaplan-Meier survival curves comparing treatment with hTRP2 and vector DNA in wild type and MHC Class I and Class II deficient mice.

FIGS. 2 A-C show mean number of surface lung metastases in mice immunized with various treatments.

FIG. 3 shows surface lung metastases in mice immunized with hTRP2 comparing wild type, IL4−/− and IFN-gamma−/−.

FIG. 4 shows abdominal depigmentation of mice abdominal quadrants over time.

FIGS. 5A-D show surface lung metastases in wild type mice, and IFN-gamma−/− mice with IFN-gamma repletion at various time points.

FIG. 6 shows % cytokine positive CD4+ cells in wild type, IL4−/− and IFN-gamma−/− after immunization.

### DETAILED DESCRIPTION OF THE INVENTION

The present invention provides a method for stimulating an immune response to a tissue expressing TRP2 in a subject individual. The subject individual is preferably human, although the invention can be applied in veterinary applications to animal species, preferably mammalian (for example horse, dog or cat) or avian species, as well.

As used in the specification and claims of this application, the term “immune response” encompasses both cellular and humoral immune responses. Preferably, the immune response is sufficient to provide immunoprotection against growth of tumors expressing TRP2. The term “stimulate” refers to the initial stimulation of a new immune response or to the enhancement of a pre-existing immune response.

In accordance with the invention, a subject individual is treated to stimulate an immune response to endogenous TRP2 by administering a xenogeneic or xenoexpressed TRP2 antigen. The term “xenogeneic” denotes the fact that the administered antigen has a sequence peptide different from the TRP2 of the species being treated and originates from a different species. For treatments of humans, preferred xenogeneic antigens will be rodent antigens, for example mouse, but could come from other mammals such as dog, cat, cow, or sheep, or from birds, fish, amphibian, reptile, insect or other more distantly related species. The term “xenoexpressed” refers to an antigen which may be syngeneic with the subject individual, but which is expressed in cells of a species different from the subject individual, for example in insect cells.

The term “TRP2 antigen” refers to a protein/peptide antigen or to a polynucleotide having a sequence that is expressed in vivo to produce the protein/peptide antigen. In either case, the protein/peptide antigen may be the entire TRP2 molecule, or some antigenic portion thereof derived from the extracellular domain. For example, plasmids are prepared using either full length cDNA or using a truncated portion encoding an amino acid strand.

Administration of a protein/peptide xenogeneic or xenoexpressed TRP2 antigen can be accomplished by several routes. First, the xenogeneic TRP2 may be administered as part of a vaccine composition which may include one or more adjuvants such as alum, QS21, TITERMAX or its derivatives, incomplete or complete Freund's and related adjuvants, and cytokines such as granulocyte-macrophage colony stimulating factor (GM-CSF), flt-3 ligand, interleukin-2, interleukin-4 and interleukin-12 for increasing the intensity of the immune response. The vaccine composition may be in the form of xenogeneic TRP2 antigen in a solution or a suspension, or the TRP2 antigen may be introduced in a lipid carrier such as a liposome. Such compositions will generally be administered by subcutaneous, intradermal or intramuscular route. Vaccine compositions containing protein/peptide xenogeneic or xenoexpressed TRP2 antigen are administered in amounts which are effective to stimulate an immune response to the target differentiation antigen in the subject individual. The preferred amount to be administered will depend on the species of the target individual and on the specific antigen, but can be determined through routine preliminary tests in which increasing doses are given and the extent of antibody formation or T cell response is measured by enzyme-linked immunosorbent assay (ELISA) or similar tests. T cell responses may also be measured by cellular immune assays, such as cytokine release assays and proliferation assays.

Xenogeneic TRP2 antigen may also be introduced in accordance with the invention using a DNA immunization technique in which DNA encoding the antigen is introduced into the subject such that the antigen is expressed by the subject. Xenogeneic TRP2 antigen may also be administered as a purified protein. Proteins can be purified for this purpose from cell lysates using column chromatography procedures. Proteins for this purpose may also be purified from recombinant sources, such as bacterial or yeast clones or mammalian or insect cell lines expressing the desired product.

Xenogeneic TRP2 antigen may also be administered indirectly through genetic immunization of the subject with DNA encoding the antigen. cDNA encoding the xenogeneic TRP2 antigen is combined with a promoter which is effective for expression of the cDNA in mammalian cells. This can be accomplished by digesting the nucleic acid polymer with a restriction endonuclease and cloning into a plasmid containing a promoter such as the SV40 promoter, the cytomegalovirus (CMV) promoter or the Rous sarcoma virus (RSV) promoter. The resulting construct is then used as a vaccine for genetic immunization. The cDNA can also be cloned into plasmid and viral vectors that are known to transduce mammalian cells. These vectors include retroviral vectors, adenovirus vectors, vaccinia virus vectors, pox virus vectors and adenovirus-associated vectors.

Xenogeneic antigen may also be administered in combination with anti-GITR (glucocorticoid-induced tumor necrosis factor receptor family gene), as described in Cohen A, et al, Agonist Anti-GITR Antibody Enhances Vaccine-Induced CD8+ T-cell Responses and Tumor Immunity, 66 CANCER RES. 4904 (2006).

The nucleic acid constructs containing the promoter, TRP2 antigen-coding region and intracellular sorting region can be administered directly or they can be packaged in liposomes or coated onto colloidal gold particles prior to administration. Techniques for packaging DNA vaccines into liposomes are known in the art, for example from Murray, ed., GENE TRANSFER AND EXPRESSION PROTOCOLS, Humana Pres, Clifton, N.J. (1991). Similarly, techniques for coating naked DNA onto gold particles are taught in Yang, Gene transfer into mammalian somatic cells in vivo, CRIT. REV. BIOTECH. 12: 335-356 (1992), and techniques for expression of proteins using viral vectors are found in Adolph, K. ed., VIRAL GENOME METHODS, CRC Press, Florida (1996).

For genetic immunization, the vaccine compositions are preferably administered intradermally, subcutaneously or intramuscularly by injection or by gas driven particle bombardment, and are delivered in an amount effective to stimulate an immune response in the host organism. The compositions may also be administered ex vivo to blood or bone marrow-derived cells (which include APCs) using liposomal transfection, particle bombardment or viral infection (including co-cultivation techniques). The treated cells are then reintroduced back into the subject to be immunized. While it will be understood that the amount of material needed will depend on the immunogenicity of each individual construct and cannot be predicted a priori, the process of determining the appropriate dosage for any given construct is straightforward. Specifically, a series of dosages of increasing size, starting at about 0.1 μg is administered and the resulting immune response is observed, for example by measuring antibody titer using an ELISA assay, detecting CTL response using a chromium release assay or detecting TH (helper T cell) response using a cytokine release assay.

In accordance with a further aspect of the present invention, an immune response against a TRP2 antigen can be stimulated by the administration of syngeneic TRP2 antigen expressed in cells of a different species, i.e. by xenoexpressed TRP2 antigen. In general, the subject being treated will be a human or other mammal. Thus, insect cells are a preferred type of cells for expression of the syngeneic differentiation antigen. Suitable insect cell lines include Sf9 cells and Schneider 2 Drosophila cells. The therapeutic differentiation antigen could also be expressed in bacteria, yeast or mammalian cell lines such as COS or Chinese hamster ovary cells. Host cells which are evolutionarily remote from the subject being treated, e.g. insects, yeast or bacteria for a mammalian subject, may be preferred since they are less likely to process the expressed protein in a manner identical to the subject.

To provide for expression of the differentiation antigen in the chosen system, DNA encoding the differentiation antigen or a portion thereof sufficient to provide an immunologically effective expression product is inserted into a suitable expression vector. There are many vector systems known which provide for expression of incorporated genetic material in a host cell, including baculovirus vectors for use with insect cells, bacterial and yeast expression vectors, and plasmid vectors (such as psvk3) for use with mammalian cells. The use of these systems is well known in the art.

For treatment of humans with a syngeneic differentiation antigen, cDNA encoding the human differentiation antigen to be targeted must be available. cDNA is produced by reverse transcription of mRNA, and the specific cDNA encoding the TRP2 antigen can be identified from a human cDNA library using probes derived from the protein sequence of the differentiation antigen, which is known in the art, examples of which are found in Seq. ID No. 1 and Seq. ID No. 2, which follow the Examples.

Xenoexpressed TRP2 antigen, like purified xenogeneic TRP2 antigen, is administered to the subject individual in an amount effective to induce an immune response. The composition administered may be a lysate of cells expressing the xenoexpressed antigen, or it may be a purified or partially purified preparation of the xenoexpressed antigen.

The invention will now be further described with reference to the following, non-limiting examples:

### Example 1

The human TRP2 (hTRP2) gene was subcloned into the PCR3 vector. The empty vector PCR3 was used as a control in some experiments. The mouse GM-CSF gene was cloned into the WRG-BEN vector.

For DNA immunization, plasmid DNA was coated on a 1-mm gold microcarrier and precipitated on bullets of Tefzel tubing. The gold-DNA complex was delivered to each abdominal quadrant of immunized animals using a helium-driven gene gun (Accell; PowderJect Vaccines, Inc.) for a total of four injections (1 mg plasmid DNA/quadrant). Immunization was repeated weekly for 3 weeks as described in the text and figure legends.

### Example 2

For intradermal tumor challenge, mice were injected on the right flank with 10̂5 B16F10LM3 melanoma cells. After palpitation and caliper measure of tumor diameter every other day, tumors were scored as present when a diameter of 2 mm was reached.

Mice were immunized with the gold-DNA complex as described above. Mice were immunized with hTRP2 DNA, control vector DNA, or left unimmunized.

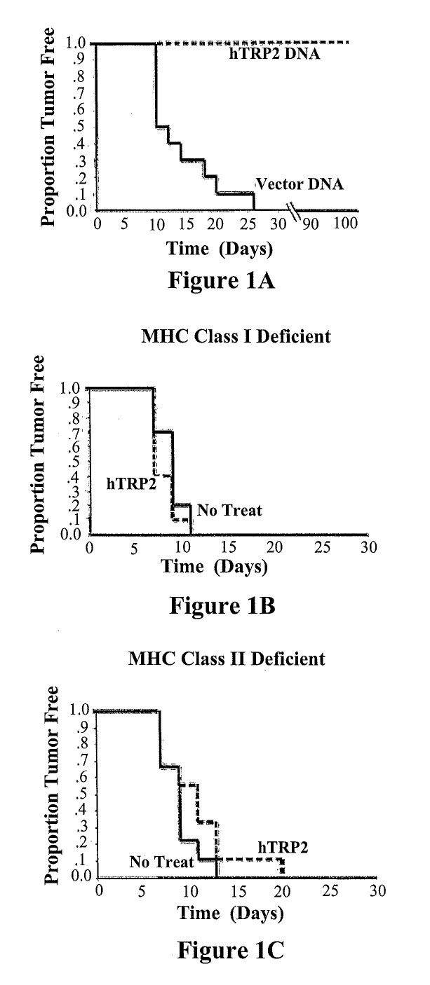

As seen in the Kaplan-Meier curves of FIGS. 1A-C tumor protection in C57BL/6 mice was observed following immunization with hTRP2 but not vector alone (FIG. 1A). No significant protection was noted in immunized mice deficient in MHC I (B) or MHC II (C), indicating a central role for both CD4+ and CD8+ T cells. Separate experiments showed that immunization did not affect the growth of established cutaneous tumors.

### Example 3

For lung metastasis challenge, mice were injected in the left rear footpad with 2×10̂5 B16F10LM3 melanoma cells selected for high spontaneous metastatic potential. The mice underwent amputation when the mean tumor size was 5+/−1 mm, approximately 19-21 days after challenge. Mice were then randomized into treatment groups (hTRP2 DNA, hTRP2+GM-CSF DNA, GM-CSF DNA) or left unimmunized. Beginning one day after resection, mice were given 3 immunizations with plasmid DNA at weekly intervals. After 23, 28, or 32 days, mice were sacrificed, lobes of the lungs were dissected, and surface lung metastases were counted.

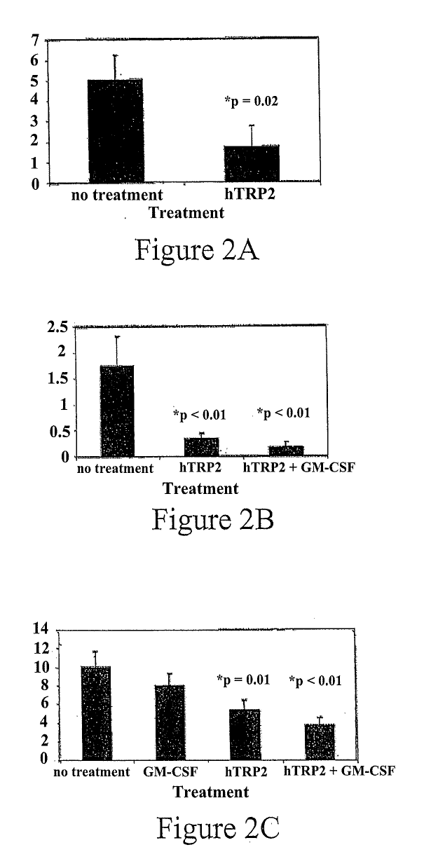

After counting of metastases, both the mean number of metastases and the number of mice that developed detectable metastases were lower in mice immunized with hTRP2 than in unimmunized controls. This is shown in FIG. 2A, the first of three experiments, where lungs were assessed 28 days after resection. In FIGS. 2A and 2B, where lungs were assessed 23 and 32 days after resection, hTRP2+GM-CSF showed lower numbers of metasases, but was not found to be significantly lower statistically (P=0.2). Table 1 shows that, if all mice from all three groups are combined, treatment with hTRP2+/−GM-CSF causes a significantly lower incidence of local recurrence.

TABLE 1
Immunization with hTRP2 Protected Mice from Local Recurrence

| | Local | Local | | | |
|---|---|---|---|---|---|
| | Recurrence | Recurrence | | | Fisher's |
| Treatment | present | absent | Total | X² | exact P |
| No | 12 | 96 | 108 | | |
| Treatment | | | | | |
| hTRP2 +/− | 2 | 127 | 129 | 9.67 | 0.002 |
| GM-CSF | | | | | |
| Total | 14 | 223 | 237 | | |

### Example 4

C57BL/6 mice were immunized by helium gun delivery of hTRP-2 coated to gold particles into each abdominal quadrant for a total of four injections. C57BL/6, IL4−/− and IFN-gamma−/− mice, all of C57BL/6 genetic background, were immunized weekly for a total of five weeks. This was followed by intravenous challenge via the tail vein with 2×10̂5 B16F10/LM3 melanoma cells performed 7 days after the final immunization. Mice were sacrificed at 14 days after tumor challenge, all lobes of both lungs were dissected, and surface lung metastases were counted under a dissecting microscope.

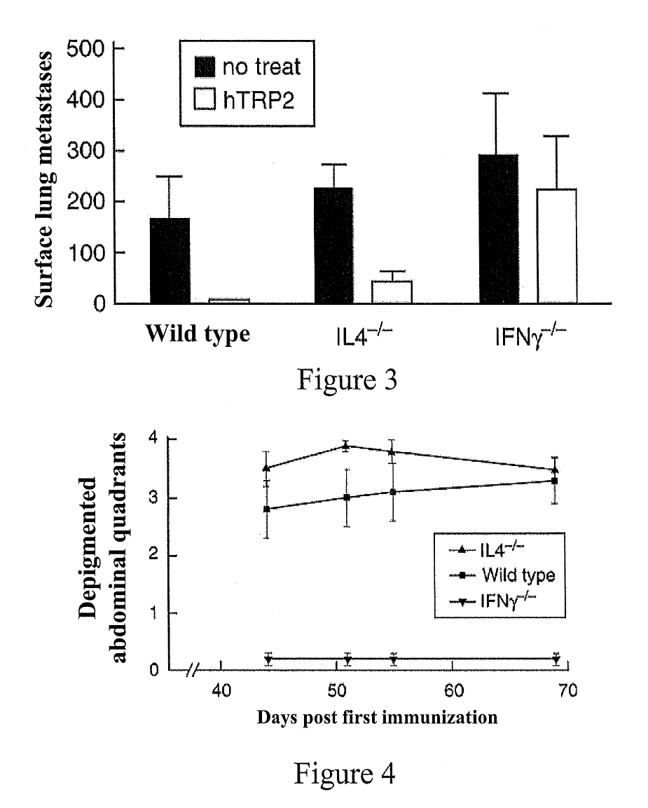

As seen in FIG. 3, immunization of wild-type mice resulted in a marked reduction in the number of surface lung metastases. Similarly, significant protection was also observed in IL4−/− mice immunized with hTRP-2. However, mice lacking the IFN-gamma gene were not protected, showing a requirement for this cytokine in tumor immunity induced by hTRP-2 DNA.

### Example 5

All mice that were immunized with the hTRP-2 gene were observed for autoimmune depigmentation for at least 70 days after starting immunization. After the final immunization, mice were shaved and depilated over the abdomen and observed for 8 weeks. Scoring of depigmentation was performed by dividing the abdomen into four quadrants. Quadrants were recorded as positive when they had an estimated >50% depigmented hairs.

Both wild type and IL4−/− mice began demonstrating evidence of depigmentation as soon as hair re-grew in previously depilated areas, between days 35-42. The rate of depigmentation was more rapid in the IL4−/− mice, compared with wild-type mice (FIG. 4). IFN-gamma−/− mice did not show any significant evidence of autoimmune depigmentation, implying that IFN-gamma is required for induction of autoimmunity as well as tumor immunity.

### Example 6

Selected IFN-gamma−/− mice were repleted with recombinant murine IFN-gamma during the entire experiment or only during the effector (tumor challenge) phase and tumor protection was assessed. Mice were repleted with 3000 U of recombinant murine IFN-gamma by intraperitoneal injection twice per week.

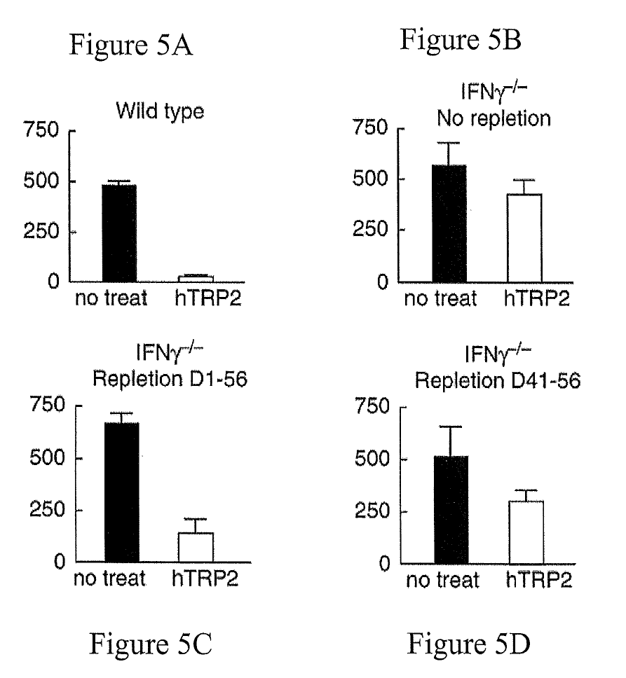

Wild type C57BL/6 mice immunized with hTRP-2 DNA were well protected (FIG. 5A). IFN-gamma−/− mice that were immunized with hTRP-2 DNA but not repleted with IFN-gamma were not protected (FIG. 5B). However, in immunized mice that were repleted with IFN-gamma during both the priming and effector phase (56 days), the number of surface lung metastases was significantly and substantially reduced compared to the knockout mice that were not repleted (P<0.0002) (FIG. 5C). In contrast, IFN-gamma−/− mice immunized with hTRP-2 DNA and repleted only during the effector phase, immediately prior to tumor cell injection, did not demonstrate significant protection from tumor challenge (FIG. 5D). Therefore the presence of IFN-gamma−/− during the effector phase alone is not sufficient to mediate significant tumor immunity.

### Example 7

CD4+ T cell lines were generated against TRP-2. Five days after the last immunization, CD4+ T lymphocytes were isolated from the spleen and draining inguinal lymph nodes by magnetic bead cell sorting (MACS). Briefly, cells were resuspended in PBS supplemented with 0.5% BSA and 2 mM EDTA, and incubated on ice for 15 minutes with anti-CD4 magnetic beads. After washing, the cells were placed on a magnetic column and two additional washes were performed. After removal from the magnet the cells were recovered and 5×10̂5 cells were co-cultured with naive irradiated (300 cGy) splenocytes (2×10′7), in RPMI 1640 (Gibco) supplemented with 10% FBS, 2 mM glutamine, 100 U/ml penicillin, 100 μg/ml streptomycin, 50 μM beta-mercaptoethanol, 0.1 mM non-essential amino acids, 1 mM sodium pyruvate and 20 μg/ml hTRP-2 237-256 peptide (sequence NESFALPYWNFATGRNECDV). Cells were restimulated weekly with peptide in media supplemented with 5% supernatant from rat lymphocytes stimulated with concanavalin A.

Monoclonal antibodies against CD3 and CD28 were used for stimulation for intracellular cytokine staining. Anti-CD4-PerCP or Cy-Chrome (clone RM4-5), anti-CD44-FITC (clone IM7) and anti-CD62L-FITC (clone MEL-14) were used for cell-surface staining. Intracellular staining for cytokines was performed using PE anti-IL4 (clone 11B11) and FITC or PE anti-IFN-gamma (clone XMG1.2). Isotype controls for the intracellular cytokines used the irrelevant rat IgG1 monoclonal R3-34 conjugated to FITC or PE. All antibodies were purchased from PharMingen. After two to three rounds of stimulation, cells were stimulated with plate-bound anti-CD3 and 2 μg/ml anti-CD28 for 6 hours, with addition of 10 μg/ml Brefeldin A for the last 2 hours. The cells were then stained for cell-surface markers and intracellular cytokines using the Cytofix/Cytoperm Kit and analyzed on a FACScalibur. Fluorescence voltages and compensation values were determined using cells single-stained with anti-CD4-FITC, anti-CD4-PE or anti-CD4-PerCP. Acquisition and analysis were performed using CellQuest software. For each tube, 30000 events were acquired in a live lymphocyte and CD4 double-gate.

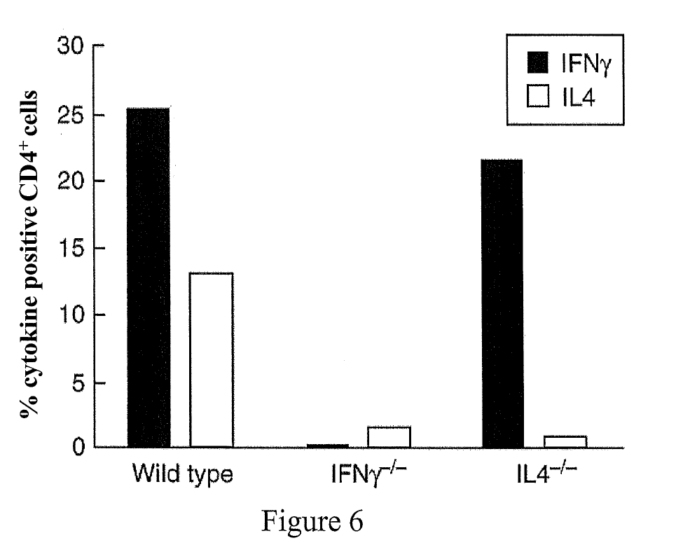

CD4+ T cell lines from wild type C57BL/6 mice immunized with hTRP-2 DNA produced both IFN-gamma and IL4 in response to the specific TRP-2 peptide (FIG. 6). CD4+ T cells from IL4−/− mice produced levels of IFN-gamma comparable to those seen in wild type mice and produced no IL4, as would be expected based on the disruption of the IL4 gene. When CD4+ T cell lines were generated from IFN-gamma−/− mice immunized with hTRP-2 DNA, as expected there is no IFN-gamma production but there was almost a 9-fold decrease in IL4 production compared to wild type mice. The significant decrease in IL4 production suggests that the lack of IFN-gamma results in a loss of ability to prime CD4+ T cells in response to hTRP2 DNA.

### Sequences

Human TRP2 cDNA was isolated and cloned in Brigitte Bouchard et al, Molecular characterization of a human tyrosinase-related-protein-2 cDNA: Patterns of expression in melanocytic cells, 219 EUR J. BIOCHEM. 127 (1994).

| Seq. ID No. 1 - human TRP2 (isoform 1) | |
|---|---|
| 1 | atgagccccc tttggtgggg gtttctgctc agttgcttgg gctgcaaaat cctgccagga |
| 61 | gcccagggtc agttcccccg agtctgcatg acggtggaca gcctagtgaa caaggagtgc |
| 121 | tgcccacgcc tgggtgcaga gtcggccaat gtctgtggct ctcagcaagg ccgggggcag |
| 181 | tgcacagagg tgcgagccga cacaaggccc tggagtggtc cctacatcct acgaaaccag |
| 241 | gatgaccgtg agctgtggcc aagaaaattc ttccaccgga cctgcaagtg cacaggaaac |
| 301 | tttgccggct ataattgtgg agactgcaag tttggctgga ccggtcccaa ctgcgagcgg |
| 361 | aagaaaccac cagtgattcg gcagaacatc cattccttga gtcctcagga aagagagcag |
| 421 | ttcttgggcg ccttagatct cgcgaagaag agagtacacc ccgactacgt gatcaccaca |
| 481 | caacactggc tgggcctgct tgggcccaat ggaacccagc cgcagtttgc caactgcagt |
| 541 | gtttatgatt tttttgtgtg gctccattat tattctgtta gagatacatt attaggacca |
| 601 | ggacgcccct acagggccat agatttctca catcaaggac ctgcatttgt tacctggcac |
| 661 | cggtaccatt tgttgtgtct ggaaagagat ctccagcgac tcattggcaa tgagtctttt |
| 721 | gctttgccct actggaactt tgccactggg aggaacgagt gtgatgtgtg tacagaccag |
| 781 | ctgtttgggg cagcgagacc agacgatccg actctgatta gtcggaactc aagattctcc |
| 841 | agctgggaaa ctgtctgtga tagcttcgat gactacaacc acctggtcac cttgtgcaat |
| 901 | ggaacctatg aaggtttgct gagaagaaat caaatgggaa gaaacagcat gaaattgcca |
| 961 | accttaaaag acatacgaga ttgcctgtct ctccagaagt ttgacaatcc tcccttcttc |
| 1021 | cagaactcta ccttcagttt caggaatgct ttggaagggt ttgataaagc agatgggact |
| 1081 | ctggattctc aagtgatgag ccttcataat ttggttcatt ccttcctgaa cgggacaaac |
| 1141 | gctttgccac attcagccgc caatgatccc atttttgtgg ttcttcattc ctttactgat |
| 1201 | gccatctttg atgagtggat gaaaagattt aatcctcctg cagatgcctg gcctcaggag |
| 1261 | ctggccccta ttggtcacaa tcggatgtac aacatggttc ctttcttccc tccagtgact |
| 1321 | aatgaagaac tctttttaac ctcagaccaa cttggctaca gctatgccat cgatctgcca |
| 1381 | gtttcagttg aagaaactcc aggttggccc acaactctct tagtagtcat gggaacactg |
| 1441 | gtggctttgg ttggtctttt tgtgctgttg gcttttcttc aatatagaag acttcgaaaa |
| 1501 | ggatatacac ccctaatgga gacacattta agcagcaaga gatacacaga agaagcctag |
| Seq. ID No. 2 - human TRP2 (isoform 2) | |
| 1 | atgagccccc tttggtgggg gtttctgctc agttgcttgg gctgcaaaat cctgccagga |
| 61 | gcccagggtc agttcccccg agtctgcatg acggtggaca gcctagtgaa caaggagtgc |
| 121 | tgcccacgcc tgggtgcaga gtcggccaat gtctgtggct ctcagcaagg ccgggggcag |
| 181 | tgcacagagg tgcgagccga cacaaggccc tggagtggtc cctacatcct acgaaaccag |
| 241 | gatgaccgtg agctgtggcc aagaaaattc ttccaccgga cctgcaagtg cacaggaaac |
| 301 | tttgccggct ataattgtgg agactgcaag tttggctgga ccggtcccaa ctgcgagcgg |
| 361 | aagaaaccac cagtgattcg gcagaacatc cattccttga gtcctcagga aagagagcag |
| 421 | ttcttgggcg ccttagatct cgcgaagaag agagtacacc ccgactacgt gatcaccaca |
| 481 | caacactggc tgggcctgct tgggcccaat ggaacccagc cgcagtttgc caactgcagt |
| 541 | gtttatgatt tttttgtgtg gctccattat tattctgtta gagatacatt attaggacca |
| 601 | ggacgcccct acagggccat agatttctca catcaaggac ctgcatttgt tacctggcac |
| 661 | cggtaccatt tgttgtgtct ggaaagagat ctccagcgac tcattggcaa tgagtctttt |
| 721 | gctttgccct actggaactt tgccactggg aggaacgagt gtgatgtgtg tacagaccag |
| 781 | ctgtttgggg cagcgagacc agacgatccg actctgatta gtcggaactc aagattctcc |
| 841 | agctgggaaa ctgtctgtga tagcttggat gactacaacc acctggtcac cttgtgcaat |
| 901 | ggaacctatg aaggtttgct gagaagaaat caaatgggaa gaaacagcat gaaattgcca |
| 961 | accttaaaag acatacgaga ttgcctgtct ctccagaagt ttgacaatcc tcccttcttc |
| 1021 | cagaactcta ccttcagttt caggaatgct ttggaagggt ttgataaagc agatgggact |
| 1081 | ctggattctc aagtgatgag ccttcataat ttggttcatt ccttcctgaa cgggacaaac |
| 1141 | gctttgccac attcagccgc caatgatccc atttttgtgg tgatttctaa tcgtttgctt |
| 1201 | tacaatgcta caacaaacat ccttgaacat gtaagaaaag agaaagcgac caaggaactc |
| 1261 | ccttccctgc atgtgctggt tcttcattcc tttactgatg ccatctttga tgagtggatg |
| 1321 | aaaagattta atcctcctgc agatgcctgg cctcaggagc tggcccctat tggtcacaat |
| 1381 | cggatgtaca acatggttcc tttcttccct ccagtgacta atgaagaact ctttttaacc |
| 1441 | tcagaccaac ttggctacag ctatgccatc gatctgccag tttcagttga agaaactcca |
| 1501 | ggttggccca caactctctt agtagtcatg ggaacactgg tggctttggt tggtcttttt |
| 1561 | gtgctgttgg cttttcttca atatagaaga cttcgaaaag gatatacacc cctaatggag |
| 1621 | acacatttaa gcagcaagag atacacagaa gaagcctag |

The cDNA for mouse TRP2 was cloned and sequenced in Jackson I J, et al, A second tyrosinase-related protein, TRP-2, maps to and is mutated at the mouse slaty locus, 11 EMBO J. 527 (1992).

| Seq. ID No. 2 - mouse TRP2 | |
|---|---|
| 1 | atgggccttg tgggatgggg gcttctgctg ggttgtctgg gctgcggaat tctgctcaga |
| 61 | gctcgggctc agtttccccg agtctgcatg accttggatg gcgtgctgaa caaggaatgc |
| 121 | tgcccgcctc tgggtcccga ggcaaccaac atctgtggat ttctagaggg cagggggcag |
| 181 | tgcgcagagg tgcaaacaga caccagaccc tggagtggcc cttatatcct tcgaaaccag |
| 241 | gatgaccgtg agcaatggcc gagaaaattc ttcaaccgga catgcaaatg cacaggaaac |
| 301 | tttgctggtt ataattgtgg aggctgcaag ttcggctgga ccggccccga ctgtaatcgg |
| 361 | aagaagccgg ccatcctaag acggaatatc cattccctga ctgcccagga gagggagcag |
| 421 | ttcttgggcg ccttagacct ggccaagaag agtatccatc cagactacgt gatcaccacg |
| 481 | caacactggc tggggctgct cggacccaac gggacccagc cccagatcgc caactgcagc |
| 541 | gtgtatgact tttttgtgtg gctccattat tattctgttc gagacacatt attaggtcca |
| 601 | ggacgcccct ataaggccat tgatttctct caccaagggc ctgcctttgt cacgtggcac |
| 661 | aggtaccatc tgttgtggct ggaaagagaa ctccagagac tcactggcaa tgagtccttt |
| 721 | gcgttgccct actggaactt tgcaaccggg aagaacgagt gtgacgtgtg cacagacgag |
| 781 | ctgcttggag cagcaagaca agatgaccca acgctgatta gtcggaactc gagattctct |
| 841 | acctgggaga ttgtgtgcga cagcttggat gactacaacc gccgggtcac actgtgtaat |
| 901 | ggaacctatg aaggtttgct gagaagaaac aaagtaggca gaaataatga gaaactgcca |
| 961 | accttaaaaa atgtgcaaga ttgcctgtct ctccagaagt ttgacagccc tcccttcttc |
| 1021 | cagaactcta ccttcagctt caggaatgca ctggaagggt ttgataaagc agacggaaca |
| 1081 | ctggactctc aagtcatgaa ccttcataac ttggctcact ccttcctgaa tgggaccaat |
| 1141 | gccttgccac actcagcagc caacgaccct gtgtttgtgg tcctccactc ttttacagac |
| 1201 | gccatctttg atgagtggct gaagagaaac aacccttcca cagatgcctg gcctcaggaa |
| 1261 | ctggcaccca ttggtcacaa ccgaatgtat aacatggtcc ccttcttccc accggtgact |
| 1321 | aatgaggagc tcttcctaac cgcagagcaa cttggctaca attacgccgt tgatctgtca |
| 1381 | gaggaagaag ctccagtttg gtccacaact ctctcagtgg tcattggaat cctgggagct |
| 1441 | ttcgtcttgc tcttggggtt gctggctttt cttcaataca gaaggcttcg caaaggctat |
| 1501 | gcgcccttaa tggagacagg tctcagcagc aagagataca cggaggaagc ctag |

All references cited herein are incorporated by reference.

We claim:

1. A method for stimulating an immune response to a tissue expressing transient receptor potential protein II (TRP2) in a subject individual of a first species, comprising administering to the subject individual an immunologically-effective amount of xenogeneic or xenoexpressed TRP2 antigen.

2. The method according to claim 1, wherein the subject individual of the first species is human.

3. The method of claim 1, wherein the TRP2 antigen is a xenogeneic TRP2 antigen derived from a source selected from the group consisting of rodents, dogs, cats, cows, and sheep TRP2 antigen.

4. The method of claim 1, wherein the step of administering is achieved by immunization with DNA encoding a xenogeneic TRP2 antigen.

5. The method of claim 4, wherein the DNA immunization is achieved by immunization with liposomes comprising DNA encoding the xenogeneic TRP2 antigen.

6. The method of claim 4, wherein the DNA immunization is achieved by immunization with gold particles coated with DNA encoding the xenogeneic TRP2 antigen.

7. The method of claim 4, wherein the DNA encoding the TRP2 antigen is an expression vector encoding the TRP2 antigen.

8. The method of claim 1 wherein the immune response is a cellular or humoral response.

9. The method of claim 8 wherein the amount of xenogeneic or xenoexpressed TRP2 antigen is sufficient to provide immunoprotection against growth of tumors expressing TRP2.

10. The method of claim 1 wherein the amount of xenogeneic or xenoexpressed TRP2 antigen is sufficient to provide immunoprotection against growth of tumors expressing TRP2.

11. The method of claim 1, wherein the step of administering is achieved by immunization with a construct comprising DNA encoding a xenogeneic TRP2 antigen, said construct resulting in expression of the xenogeneic TRP2 antigen in the subject individual.

12. The method of claim 1, wherein the subject individual has melanoma.

13. The method of claim 12, wherein the step of administering is achieved by immunization with a construct comprising DNA encoding a xenogeneic TRP2 antigen, said construct resulting in expression of the xenogeneic TRP2 antigen in the subject individual.


SEQUENCE LISTING (3 sequences)

SEQ ID NO: 1 - DNA, 1560 bp, Homo sapiens
atgagccccctttggtgggggtttctgctcagttgcttgggctgcaaaatcctgccaggagcccagggtcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactctcttagtagtcatgggaacactggtggctttggttggtctttttgtgctgttggcttttcttcaatatagaagacttcgaaaaggatatacacccctaatggagacacatttaagcagcaagagatacacagaagaagcctag

SEQ ID NO: 2 - DNA, 1659 bp, Homo sapiens
atgagccccctttggtgggggtttctgctcagttgcttgggctgcaaaatcctgccaggagcccagggtcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggtgatttctaatcgtttgctttacaatgctacaacaaacatccttgaacatgtaagaaaagagaaagcgaccaaggaactcccttccctgcatgtgctggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactctcttagtagtcatgggaacactggtggctttggttggtctttttgtgctgttggcttttcttcaatatagaagacttcgaaaaggatatacacccctaatggagacacatttaagcagcaagagatacacagaagaagcctag

SEQ ID NO: 3 - DNA, 1554 bp, Mus musculus
atgggccttgtgggatgggggcttctgctgggttgtctgggctgcggaattctgctcagagctcgggctcagtttccccgagtctgcatgaccttggatggcgtgctgaacaaggaatgctgcccgcctctgggtcccgaggcaaccaacatctgtggatttctagagggcagggggcagtgcgcagaggtgcaaacagacaccagaccctggagtggcccttatatccttcgaaaccaggatgaccgtgagcaatggccgagaaaattcttcaaccggacatgcaaatgcacaggaaactttgctggttataattgtggaggctgcaagttcggctggaccggccccgactgtaatcggaagaagccggccatcctaagacggaatatccattccctgactgcccaggagagggagcagttcttgggcgccttagacctggccaagaagagtatccatccagactacgtgatcaccacgcaacactggctggggctgctcggacccaacgggacccagccccagatcgccaactgcagcgtgtatgacttttttgtgtggctccattattattctgttcgagacacattattaggtccaggacgcccctataaggccattgatttctctcaccaagggcctgcctttgtcacgtggcacaggtaccatctgttgtggctggaaagagaactccagagactcactggcaatgagtcctttgcgttgccctactggaactttgcaaccgggaagaacgagtgtgacgtgtgcacagacgagctgcttggagcagcaagacaagatgacccaacgctgattagtcggaactcgagattctctacctgggagattgtgtgcgacagcttggatgactacaaccgccgggtcacactgtgtaatggaacctatgaaggtttgctgagaagaaacaaagtaggcagaaataatgagaaactgccaaccttaaaaaatgtgcaagattgcctgtctctccagaagtttgacagccctcccttcttccagaactctaccttcagcttcaggaatgcactggaagggtttgataaagcagacggaacactggactctcaagtcatgaaccttcataacttggctcactccttcctgaatgggaccaatgccttgccacactcagcagccaacgaccctgtgtttgtggtcctccactcttttacagacgccatctttgatgagtggctgaagagaaacaacccttccacagatgcctggcctcaggaactggcacccattggtcacaaccgaatgtataacatggtccccttcttcccaccggtgactaatgaggagctcttcctaaccgcagagcaacttggctacaattacgccgttgatctgtcagaggaagaagctccagtttggtccacaactctctcagtggtcattggaatcctgggagctttcgtcttgctcttggggttgctggcttttcttcaatacagaaggcttcgcaaaggctatgcgcccttaatggagacaggtctcagcagcaagagatacacggaggaagcctag